SEQUENCE LISTING (2 sequences)

SEQ ID NO: 1 - protein, 443 aa, Homo sapiens
MDSRAQLWGLALNKRRATLPHPGGSTNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGDWIFTIREKDPKNLENGALQPSDLDRNKMKDIPKRPFSQCLSTIISPLFAELKEKSQACGGNLGSIEELRGAIYLAEEACPGISDTMVAQLVQRLQRYSLSGGGTSSH

SEQ ID NO: 2 - protein, 431 aa, Homo sapiens
MAHSPVQSGLPGMQNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGDWIFTIREKDPKNLENGALQPSDLDRNKMKDIPKRPFSQCLSTIISPLFAELKEKSQACGGNLGSIEELRGAIYLAEEACPGISDTMVAQLVQRLQRYSLSGGGTSSH

(19) United States
(12) Patent Application Publication
(10) Pub. No.: US 2013/0089538 A1
(43) Pub. Date: Apr. 11, 2013

(54) TREATING CANCER BY MODULATING MAMMALIAN STERILE 20-LIKE KINASE 3

(71) Applicants: Hauke Hermann Karl Cornils, Brookline, MA (US); Brian Arthur Hemmings, Bettingen (CH); Mario Reinhard Stegert, Riehen (CH)

(72) Inventors: Hauke Hermann Karl Cornils, Brookline, MA (US); Brian Arthur Hemmings, Bettingen (CH); Mario Reinhard Stegert, Riehen (CH)

(73) Assignee: Novartis Forschungsstiftung, Zweigniederlassung Friedrich Miescher Institute forBiomedical Researh, Basel (CH)

(21) Appl. No.: 13/702,726

(22) PCT Filed: Jun. 9, 2011
(86) PCT No.: PCT/EP2011/059570

§ 371 Date: Dec. 7, 2012

(30) Foreign Application Priority Data
Jun. 10, 2010 (EP) 10165582.7

(51) Int. Cl. A61K 45/06; A61K 31/713; A61K 39/395

(52) U.S. Cl. CPC A61K 45/06; A61K 39/3955; A61K 31/713
USPC 424/130.1; 514/44 A; 435/375; 435/6.13; 435/19

(57) ABSTRACT

The present invention relates to a method for modulating miRNA, said method being characterized in that a modulator of XRN1 is used. Also provided are uses of said method for therapeutical purposes, reagents therefore, as well as screening methods.

### FIELD OF THE INVENTION

The present invention relates to a method of treating cancer by modulating Mammalian Sterile 20-like Kinase 3.

### BACKGROUND OF THE INVENTION

Cancers and malignant tumors are characterized by continuous cell proliferation and cell death and are related causally to both genetics and the environment. Genes whose expression are associated with cancer, and the products of said genes, are of potentially great importance as cancer markers in the early diagnosis and prognosis of various cancers, as well as potential targets in cancer treatment. Cancer is a leading cause of human death next to coronary disease. In the United States, cancer causes the death of over half-million people each year and about two million new cases of cancer are diagnosed each year. The identification of new genes essential for the growth of tumors has been an objective of cancer research over the past several decades.

In order to duplicate, cells, cancer cells included, have to proceed through a defined order of events collectively called the cell cycle. The eukaryotic cell cycle consists of 4 phases, which can roughly be defined by growth and preparation for the duplication of the genetic material in G1-phase, duplication of the genetic material in S-phase, preparation for separation in G2-phase and finally separation of the genetic material into two daughter cells in M-phase. Proper progression through a given cell cycle phase and unidirectional transition between the phases are highly controlled on multiple levels (J. Massague, Nature 432, 298-306 (2004)). Defects in the mechanisms controlling the cell cycle have been shown to result in accumulation of genetic alterations and subsequent cancer development (M. Malumbres, M. Barbacid, Nat Rev Cancer 9, 153-166 (2009)). The G1-phase of the cell cycle is a very important integrator of internal and external cues, allowing cells to grow, process outside information or repair damage before entering S-phase. In G1 a cell decides whether to self renew, differentiate or die. Entry into S-phase is mediated by the action of Cyclin-Cdk complexes. Initially Cyclin D-Cdk4/6 and later Cyclin E-Cdk2 complexes phosphorylate the Rb tumor suppressor protein allowing dissociation of Rb from E2F transcription factors and subsequent transcription of genes required for S-phase entry (J. W. Harbour, et al., Cell 98, 859-869 (1999)).

The activity of Cyclin-dependent kinases is controlled on multiple levels (C. J. Sherr, J. M. Roberts, Genes Dev 18, 2699-2711 (2004)). The association of Cdks with Cylin subunits is a prerequisite for Cdk activation. This process is controlled firstly by the availability of the Cyclin sub-unit, which abundance is regulated both by transcriptional and post-transcriptional processes. Furthermore, Cyclin-Cdk inhibitor (CKI) proteins of the Cip/Kip and INK4 family control Cyclin-Cdk activity by different mechanisms. Whereas members of the Cip/Kip family such as p21, p27 and p57 directly inhibit the Cdk activity, the INK4 family members p15, p16, p18 and p19 actively promote Cyclin-Cdk complex disassembly. Interestingly, the Cip/Kip family members p21 and p27 are needed for efficient assembly of Cyclin D-Cdk4/6 complexes (C. J. Sherr, J. M. Roberts, Genes Dev 13, 1501-1512 (1999)). Importantly, loss of several CKI proteins has been associated with tumor development (M. Malumbres, M. Barbacid, Nat Rev Cancer 9, 153-166 (2009)).

Members of the NDR family of Ser/Thr kinases are highly conserved from yeast to men and have been implicated in the regulation of a variety of biological processes (A. Hergovich et al., Nat Rev Mol Cell Biol 7, 253-264 (2006)). With the regulation of mitotic exit, cell growth, proliferation, centrososme duplication and morphogenesis, NDR kinases across species have been shown to function in processes tightly linked to the cell cycle. The human genome encodes four different NDR kinase family members: NDR1/2 and LATS1/2 (A. Hergovich et al., Biochim Biophys Acta 1784, 3-15 (2008)). The kinases LATS1/2 function as part of the HIPPO pathway thereby controlling the localization and function of the YAP oncogene (B. Zhao, et al., Curr Opin Cell Bio/20, 638-646 (2008)). The HIPPO pathway thereby controls cell growth, cell size, proliferation and apoptosis. Furthermore, roles for LATS1 and LATS2 in controlling mitotic exit and genomic stability have been described (J. Bothos et al., Cancer Res 65, 6568-6575 (2005); J. P. McPherson et al., Embo J23, 3677-3688 (2004)).

Although well characterized in terms of biochemical regulation, functions for the other two NDR family kinases in the human genome NDR1 and NDR2 only recently started to unravel. In cellular systems NDR kinases have been implicated in the regulation of centrosome duplication, apoptosis and the alignment of mitotic chromosomes (S. Chiba et al., Curr Biol 19, 675-681 (2009); A. Hergovich et al., Mol Cell 25, 625-634 (2007); A. Vichalkovski et al., Curr Biol 18, 1889-1895 (2008)). Furthermore, a recent, yet unpublished, study of some of the inventors indicated a tumor suppressive function for NDR1/2 in mice by controlling proper apoptotic responses. Interestingly, with the involvement of MST1 and hMOB1 in the regulation of NDR1/2 in centrosome duplication and apoptosis and MST2 in the alignment of mitotic chromosomes, several components of the HIPPO pathway also function in the regulation of NDR1/2 (A. Hergovich et al., Curr Biol 19, 1692-1702 (2009)). However, although first functions for NDR1/2 were defined recently, downstream signaling remained elusive. Furthermore, although NDR kinases have been implicated in cell-cycle dependent processes such as centrosome duplication and the alignment of mitotic chromosomes, discrepancies exist whether NDR kinases are activated in M or S-phase of the cell cycle.

Therefore, the present inventors investigated the activation of NDR1/2 throughout the cell cycle. They surprisingly show that NDR1/2 are activated in G1-phase by MST3, the third MST-family kinase shown to function upstream of NDR1/2. In addition, with the direct regulation of c-myc and p21 stability, the present inventors defined first downstream signaling mechanisms by which NDR kinases control G1-progression and S-phase entry.

### SUMMARY OF THE INVENTION

NDR kinases have been shown to function as tumor suppressors in T-cell lymphoma. In this context, NDR kinases are regulated by the HIPPO-pathway components MST1 and MST2. The present inventors show for the first time that NDR is directly regulated by MST3 at a defined phase of the cell cycle. Furthermore they provide evidence that a MST3-NDR axis positively regulates the stability of the proto-oncogene c-myc and promotes S-phase entry. These results surprisingly clearly link MST3 to cancer development.

The present invention hence provides a method for treating cancer in a subject by modulating MST3 via the administration of a therapeutically effective amount of a modulator of MST3 to said subject. In some embodiments, MST3 is modulated by an inhibitor, such as an antibody. Alternatively, the inhibitor decreases or silences the expression of MST3, and is for instance a siRNA. In some embodiments, the subject is a mammal, for instance a human subject.

The methods of the invention are suitable for all cancers dependent on the activity of MST3. In one embodiment, the HIPPO-pathway is misregulated in the cells of the cancer. Examples of such a misregulation are mutations, amplifications or overexpression resulting in a decrease in HIPPO pathway activity and in increased activity of the YAP proto-oncogene. In one embodiment, the cancer is a lung adenocarcinoma, a prostate adenocarcinoma, a breast ductal cancer or a breast carcinoma, NOS. In some embodiments, the cancer is treated by inhibiting/reducing metastasis formation.

The present invention also encompasses a siRNA decreasing or silencing the expression of MST3 or an antibody specifically binding to MST3 for use as a medicament to treat cancer. Alternatively, the antibody can inhibit the interaction between of MST3 and one of its partners.

The present invention also provides methods of screening for agents able to modulate the expression of MST3 expression and/or biological activity, which method comprises: (i) contacting a MST3 polypeptide or a fragment thereof having the biological activity of MST3, a polynucleotide encoding such a polypeptide or polypeptide fragment, an expression vector comprising such a polynucleotide or a cell comprising such an expression vector, and a test substance under conditions that in the absence of the test substance would permit MST3 expression and/or biological activity; and (ii) determining the amount of MST3 expression and/or biological activity, to determine whether the test substance modulates MST3 biological activity and/or expression, wherein a test substance which modulates MST3 biological activity and/or expression is a potential therapeutical agent to treat cancer. An example of the biological activity of MST3 is its interaction with NDR.

In addition, the present invention also encompasses a method of diagnosing cancer comprising the step of assessing the level of expression of MST3 in a sample from a subject.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1: NDR kinases are activated by MST3 in G1 phase of the cell cycle. (a) NDR kinases are activated in a cell cycle-dependent manner. Synchronized HeLa S3 cells were harvested after mitotic shake-off and replated in fresh medium for the indicated time. Activation of NDR1/2 was assessed using anti-T444-P, NDR1 and NDR2 antibodies. Cell cycle distribution was assessed using propidium iodide (P1) staining and FACS analysis. (b) Endogenous NDR kinase activity is increased in G1 phase. HeLa cells were arrested at G2/M-border using nocodazole treatment for 14 h and released for the indicated time before harvesting. Lysates were subjected to immunoblotting and immunoprecipitation of endogenous NDR species using a mixture of isoform specific antibodies. NDR kinase activity was assessed using peptide kinase assay (n=3; P<0.002). (c) Expression of DN-MST3 reduces G1-activation of NDR. HeLa cells were transfected with dominant-negative MST1, MST2 or MST3 and 24 h later arrested with nocodazole for 14 h. Arrested cells were harvested or released into G1 for 8 h before harvesting. NDR activation was assessed using T-444-P antibody. Cell cycle phases were confirmed by analyzing Cyclin B1 and p27 expression. (d) Reduction of MST3 impairs G1-activation of NDR. HeLa cells were transfected with control siRNA (siC) or siRNA against MST3 (siMST3) and treated and analyzed as described in C. MST3 activation was assessed using a P-MST4-T178/-MST3-T190/-STK25-T174 specific antibody (anti-P-MST3). Note that the P-MST3 signal disappears in the siMST3-treated samples.

FIG. 2: shRNA-mediated knock-down of NDR1/2 results in cellular proliferation defects due to a G1-block. (a) Characterization of T-Rex-HeLa cells stably expressing shRNA against NDR1 and NDR2. Cells were seeded in 10 cm dishes and shRNA expression was induced by the addition of tetracycline (TET) for the indicated time. Lysates from harvested cells were analyzed for NDR1 and NDR2 expression using isoform-specific antibodies (*, unspecific band). (b) NDR1/2 depletion results in proliferation defects. HeLa cells expressing shRNA against NDR1/2 (shNDR1/2) or firefly luciferase (shLUC) as a control were seeded in triplicates and tetracycline was added to induce shRNA expression. After the indicated time, cells were harvested by trypsination and counted using a Vicell-automated cell counter. (c) Validation of proliferation defects in different clones stably expressing shNDR1/2 or shLUC (n=3; P<0.001). Experiments were performed as in (b), differences in proliferation were calculated as percentage of cells without tetracycline to cells with tetracycline counted on day 6 after induction of shRNA expression. (d) Depletion of NDR1/2 results in G1-arrest. Knock-down of NDR1/2 was induced for 4 days using tetracycline. 14 h before harvesting and processing for FACS analysis, cells were treated with 2.5 μg/ml nocodazole to induce G2/M accumulation. Fixed cells were stained with PI and analyzed by FACS. Histograms were overlaid to facilitate for better comparison of cells in a given cell cycle phase. (e) Reduced NDR kinase levels increase p21, p27 and decrease c-myc. Knock-down of NDR1/2 was induced for 4 days and cell lysates were analyzed for the expression of the indicated cell cycle regulators using western blotting. (f) NDR1/2 depletion does not impact c-myc mRNA levels. Knock-down of NDR1/2 was induced for the indicated time points and extracts were prepared to analyze c-myc mRNA by qPCR and protein levels by western blotting. Values are given as fold change to untreated samples (n=3).

FIG. 3: NDR1/2 in an active conformation stabilize c-myc by interfering with Skp2 and FBW7 mediated ubiquitination. (a) c-myc binds to the N-terminal region of NDR1. HEK293 cells were transfected with c-myc together with the indicated HA-tagged NDR1 constructs. NDR1 species were immunoprecipitated and c-myc binding was analyzed by SDS-PAGE. (b) Binding of NDR1 to c-myc is modulated by hydrophobic phosphorylation (T444). HEK293 cells were transfected with the indicated NDR1 contructs (NDR1TA=T444A; NDR1-3×A=T74, S281, T444A). c-myc was immunoprecipitated and bound NDR1 species were analyzed by immunoblotting. (c) Overexpression of NDR1 wt and NDR1 kd stabilizes c-myc. HEK293 cells were transfected with c-myc and the indicated NDR1 cDNA. 24 h later cells were treated with cycloheximide (CHX) for the indicated time and c-myc levels were analyzed using the LI-COR Odyssey system. (d) Hydrophobic motif phosphorylation of NDR increases binding of NDR to c-myc. HEK293 cells were transfected with c-myc and the indicated NDR1 cDNAs together with a vector encoding FLAG-MST3 and complex formation was analyzed. (e) Hydrophobic-motif phosphorylation of NDR increases c-myc stability. HEK293 cells transfected with c-myc and NDR1 constructs in the presence of absence of FLAG-MST3 were treated with CHX for 120 min and lysates were analyzed for the expression of c-myc, as compared to untreated samples, using the LI-COR Odyssey system. (f) NDR overexpression promotes c-myc dependent transcription. HEK293 cells were transfected with the indicated constructs and c-myc dependent transcription of the LDH-A promoter was analyzed (n=3; P<0.001). (g) NDR overexpression stabilizes endogenous c-myc levels. HEK293 cells were transfected with HA-tagged variants of NDR1 together with HA-MST3 where indicated and lysates were analyzed for the expression of endogenous c-myc 24 h later (*, HA-MST3; **, HA-NDR). (h) NDR impairs c-myc ubiquitination. HEK293 cells were transfected with c-myc and His-tagged ubiquitin (His-Ub) together with HA-NDR1 wt where indicated. Ubiquitinated proteins were pulled-down from cell lysates using Ni-NTA sepharose and analyzed by SDS-PAGE. (i) NDR decreases FBW7 mediated ubiquitination of c-myc. Experiment was performed as in (h), but where indicated gfp-FBW7 was co-expressed. (j) NDR impairs Skp2 mediated ubiquitination. Experiment was performed as in (h), but where indicated HA-gfp-Skp2 was co-expressed.

FIG. 4: Overexpression of NDR1/2 promotes S phase entry by stabilizing c-myc. (a) The effects of NDR kinase depletion can be rescued depending on the NDR mutant. HeLa cells expressing shRNA against NDR2 were treated for 48 h with tetracycline and subsequently transfected with the indicated NDR2 mutants refractory to shNDR2 (*, HA-tagged NDR2). (b) Overexpression of NDR stabilizes c-myc in G1. Rat1 cells were arrested in G0 by serum deprivation and released for the indicated time (* HA-NDR). (c) Overexpression of NDR promotes S phase entry. Rat-1 cells were treated as described in (b) and cell cycle distribution was analyzed using PI-staining (n=3). (d) Overexpression of NDR increases the abundance of the mycER fusion protein. Rat-1-mycER cells overexpressing HA-NDR1 wt were analyzed for the expression of NDR1 and the mycER fusion protein. (e) Overexpression of NDR increases c-myc mediated cell cycle entry. Rat-1-mycER cells were serum-arrested either in the presence or absence of 4-hydroxytamoxifen (4-OHT) (200 nM) for 48 h. Cell cycle distribution was assessed using PI staining (n=3; P<0.002).

FIG. 5: Expression of STK24/MST3 in various cancer types as retrieved from genesapiens.org MST3 seems to be overexpressed at least in subtypes of lung, breast and prostate cancers.

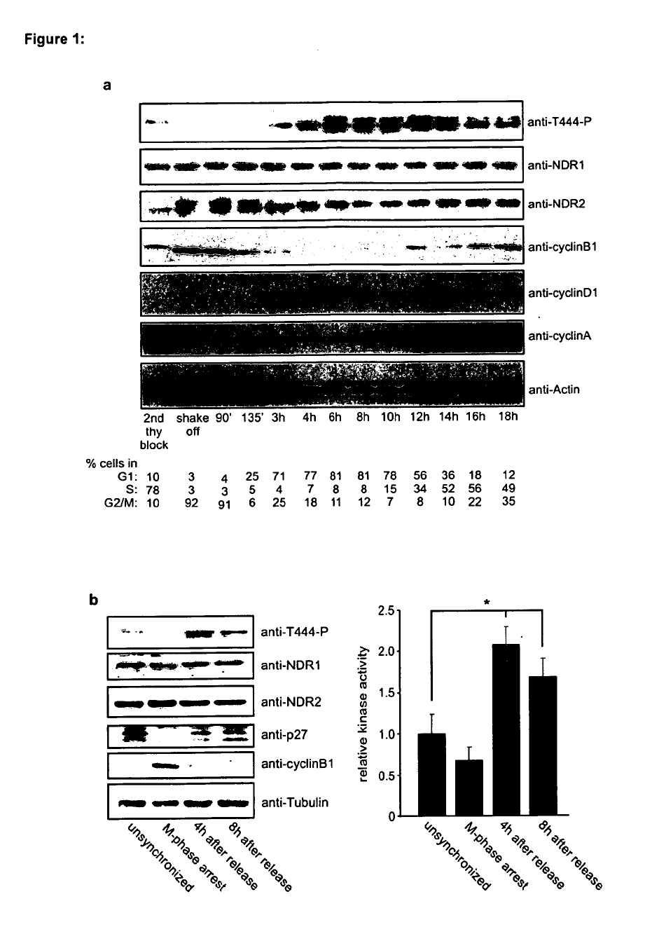

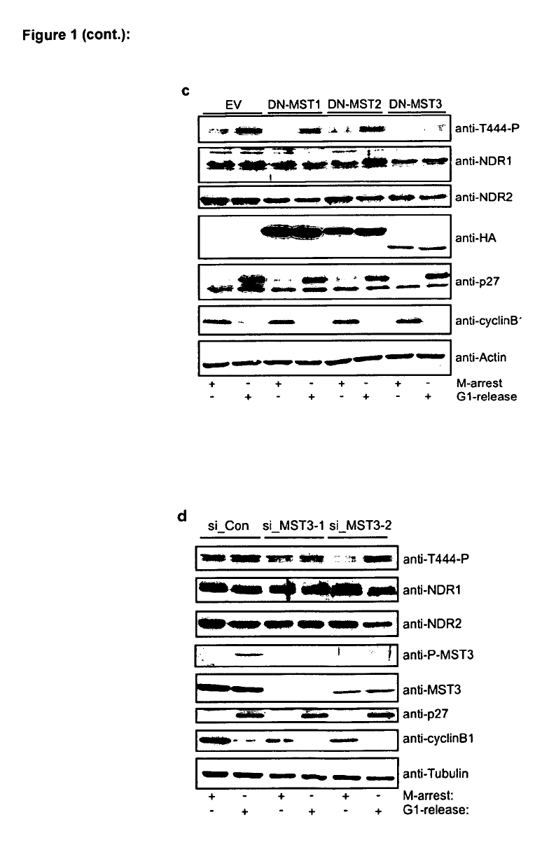

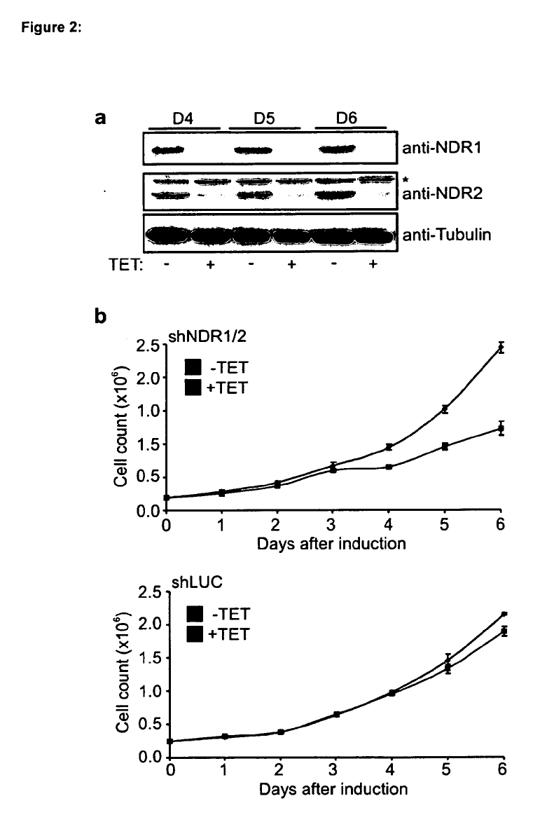

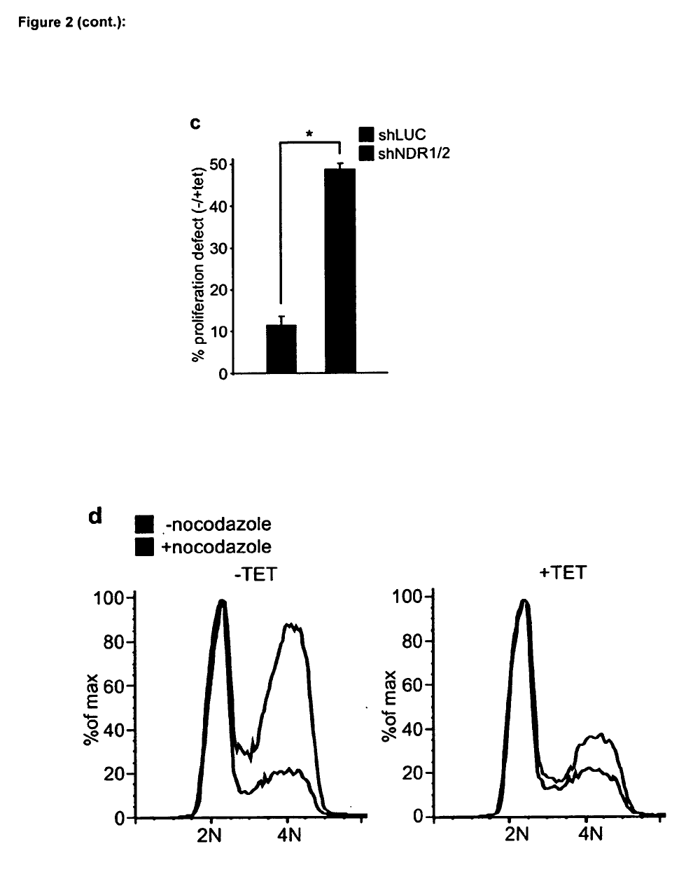

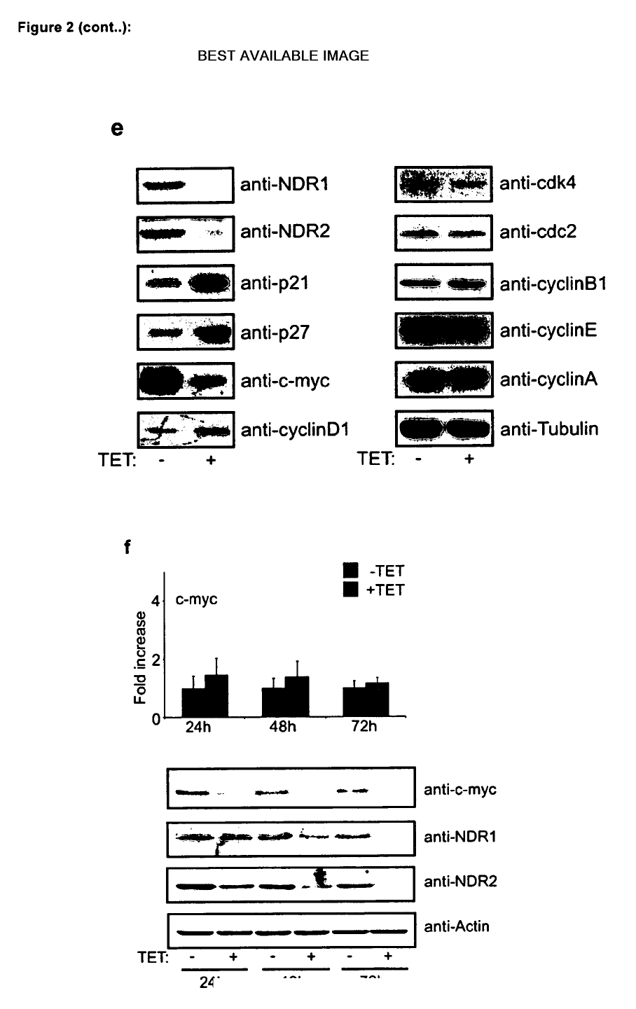

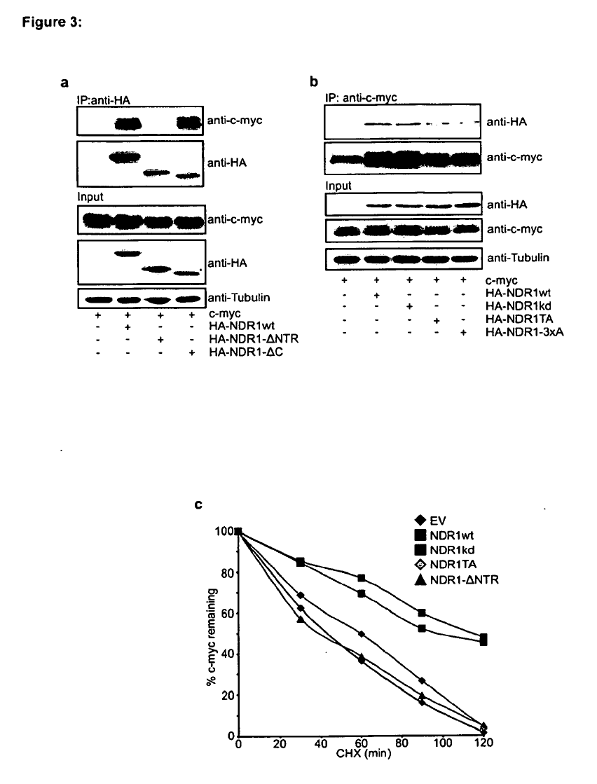

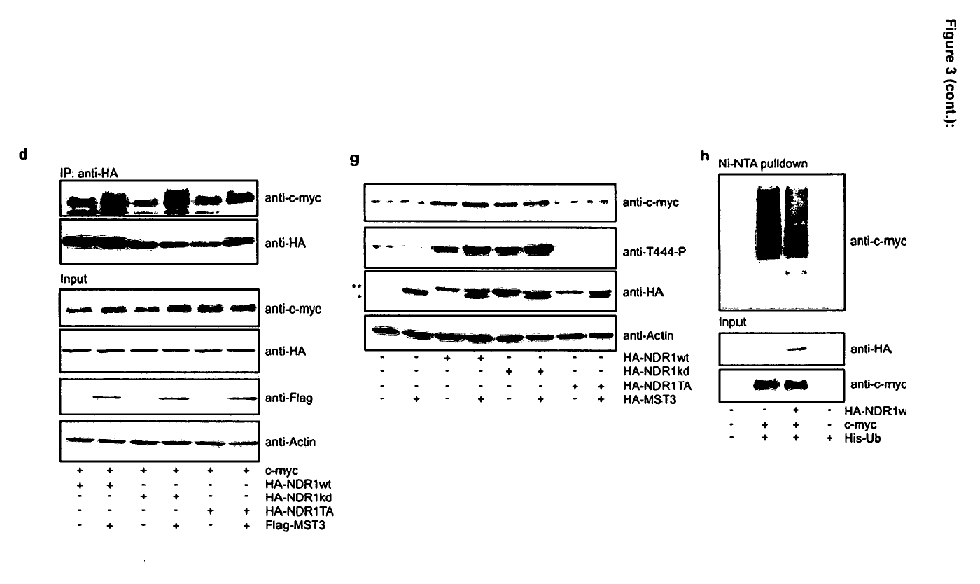

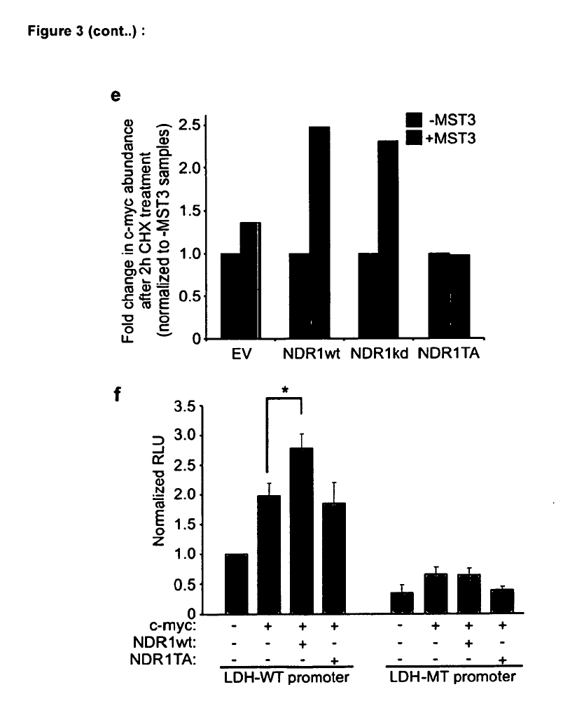

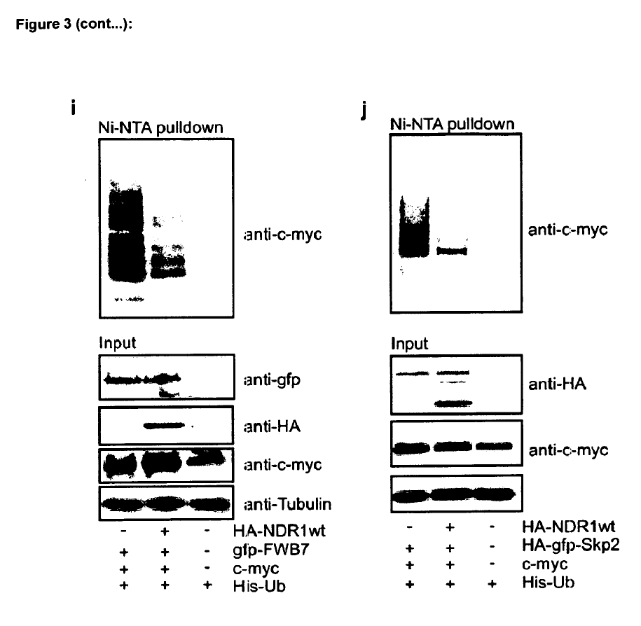

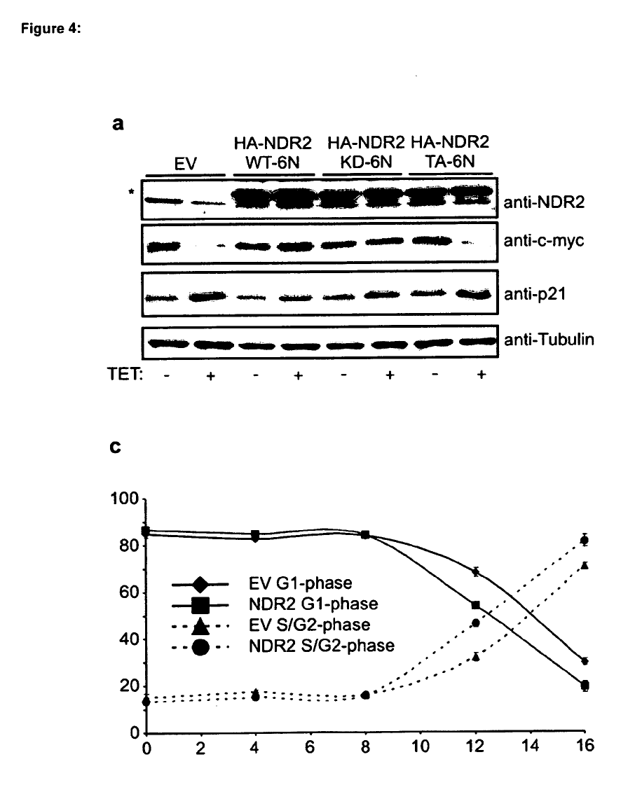

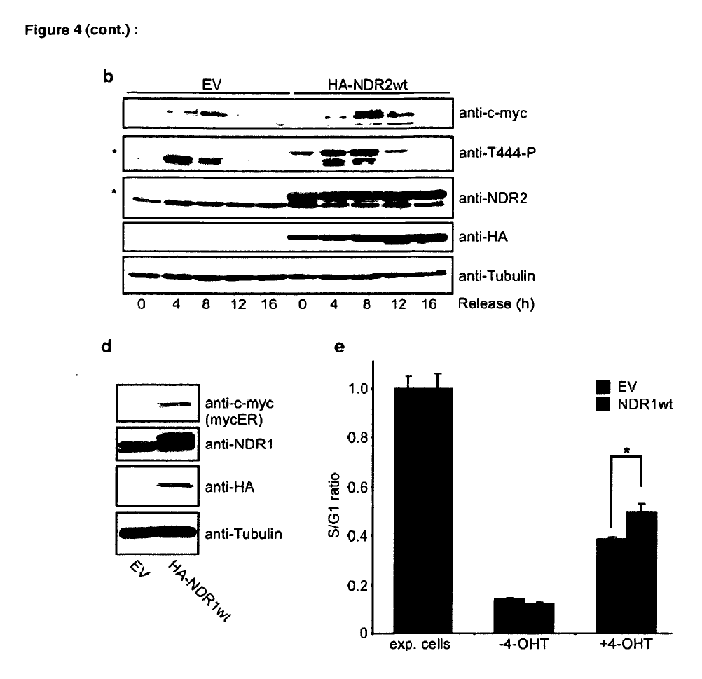

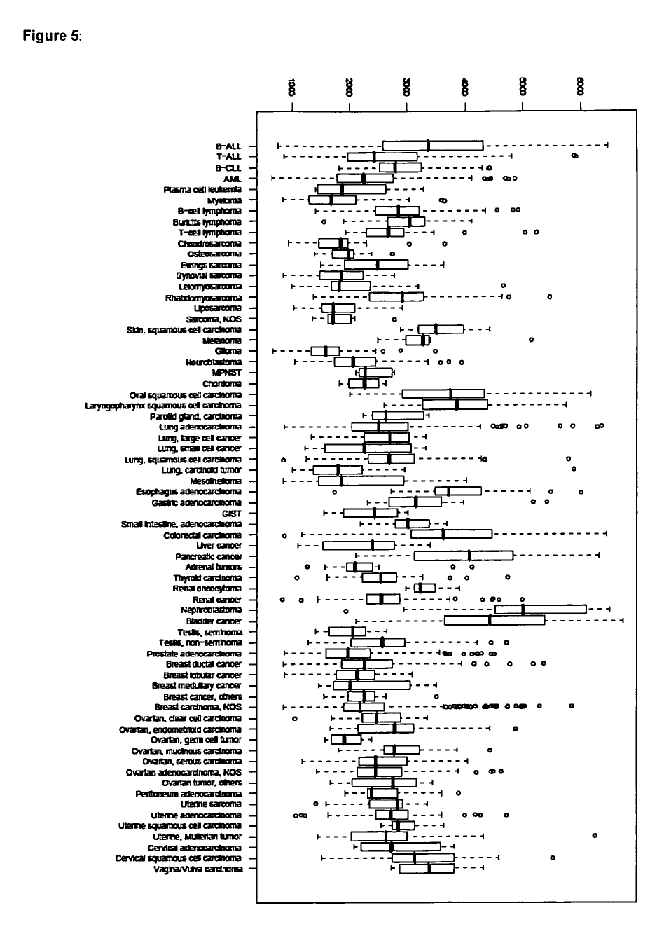

### DETAILED DESCRIPTION OF THE INVENTION

NDR kinases have been shown to function as tumor suppressors in T-cell lymphoma. In this context, NDR kinases are regulated by the HIPPO-pathway components MST1 and MST2. The present inventors show for the first time that NDR is, surprisingly, directly regulated by MST3 at a defined phase of the cell cycle. Furthermore they provide evidence that a MST3/NDR axis positively regulates the stability of the proto-oncogene c-myc and promotes S-phase entry. These results surprisingly clearly link MST3 to cancer development.

The present invention hence provides a method for treating cancer in a subject by modulating MST3 via the administration of a therapeutically effective amount of a modulator of MST3 to said subject. In some embodiments, MST3 is modulated by an inhibitor, such as an antibody. Alternatively, the inhibitor decreases or silences the expression of MST3, and is for instance a siRNA. In some embodiments, the subject is a mammal, for instance a human subject.

The methods of the invention are suitable for all cancers dependent on the activity of MST3. In one embodiment, the HIPPO-pathway is misregulated in the cells of the cancer. Examples of such a misregulation are mutations, amplifications or overexpression resulting in a decrease in HIPPO pathway activity and in increased activity of the YAP proto-oncogene. In one embodiment, the cancer is a lung adenocarcinoma, a prostate adenocarcinoma, a breast ductal cancer or a breast carcinoma, NOS. In some embodiments, the cancer is treated by inhibiting/reducing metastasis formation.

The present invention also encompasses a siRNA decreasing or silencing the expression of MST3 or an antibody specifically binding to MST3 for use as a medicament to treat cancer. Alternatively, the antibody can inhibit the interaction between of MST3 and one of its partners.

The present invention also provides methods of screening for agents able to modulate the expression of MST3 expression and/or biological activity, which method comprises: (i) contacting a MST3 polypeptide or a fragment thereof having the biological activity of MST3, a polynucleotide encoding such a polypeptide or polypeptide fragment, an expression vector comprising such a polynucleotide or a cell comprising such an expression vector, and a test substance under conditions that in the absence of the test substance would permit MST3 expression and/or biological activity; and (ii) determining the amount of MST3 expression and/or biological activity, to determine whether the test substance modulates MST3 biological activity and/or expression, wherein a test substance which modulates MST3 biological activity and/or expression is a potential therapeutical agent to treat cancer. An example of the biological activity of MST3 is its interaction with NDR.

In addition, the present invention also encompasses a method of diagnosing cancer comprising the step of assessing the level of expression of MST3 in a sample from a subject.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO₄; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons). Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue. “Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=O, Cutoff Score=1, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

In one embodiment where one is assaying for the ability to bind or compete with full-length MST3 polypeptide for binding to MST3 antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Assays described herein and otherwise known in the art may routinely be applied to measure the ability of MST3 polypeptides and fragments, variants derivatives and analogs thereof to elicit MST3-related biological activity (either in vitro or in vivo) and/or to assess whether MST3 is present in a given sample, e.g. a sample isolated from a patient.

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In addition, in the context of the present invention, the term “antibody” shall also encompass alternative molecules having the same function, e.g. aptamers and/or CDRs grafted onto alternative peptidic or non-peptidic frames. In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992). Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387. The antibodies as defined for the present invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5, 661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above. Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)). Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art. In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies. Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)). The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide). Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating cancer in a subject by inhibiting MST3 by administration to the subject of an effective amount of an inhibitory compound or pharmaceutical composition comprising such inhibitory compound. In some embodiments, said inhibitory compound is an antibody or an siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of cancer by inhibiting a MST3. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems. For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for examplel mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein. In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity. Calculation of percentage identities between different amino acid/polypeptide/nucleic acid sequences may be carried out as follows. A multiple alignment is first generated by the ClustaIX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared.

Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustaIX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg. de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs. washington.edu/phylip.html) using the “Similarity Table” option as the model for amino acid substitution (P). For DNA/RNA, an identical option exists within the DNADIST program of PHYL1 P. The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 by and 35 bp, or between 15 by and 30 bp, for instance between 18 by and 25 bp. In some embodiments, the siRNA molecule comprises more than 20 and less than 23 bp. Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap,c) a modified internucleoside linkage; or d) a modified sugar or base moiety.

Suitable modifications include, but are not limited to modifications to the sugar moiety (i.e. the 2′ position of the sugar moiety, such as for instance 2′-O-(2-methoxyethyl) or 2′-M0E) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly. In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product). Inhibitors according to any embodiment of the present invention may be used in a monotherapy (e.g. use of siRNAs alone). However it will be appreciated that the inhibitors may be used as an adjunct, or in combination with other therapies.

The inhibitors of MST3 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The inhibitors of MST3 may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted at the site of a tumour, and the molecule may be released over weeks or months. Such devices may be particularly advantageous when long term treatment with an inhibitor of MST3 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an inhibitor of MST3 may be used for the treatment of cancer in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody. The term “cancer” refers to a group of diseases in which cells are aggressive (grow and divide without respect to normal limits), invasive (invade and destroy adjacent tissues), and sometimes metastatic (spread to other locations in the body). These three malignant properties of cancers differentiate them from benign tumors, which are self-limited in their growth and don't invade or metastasize (although some benign tumor types are capable of becoming malignant). A particular type of cancer is a cancer forming solid tumours. Such cancer forming solid tumours can be breast cancer, prostate carcinoma or oral squamous carcinoma. Other cancer forming solid tumours for which the methods and inhibitors of the invention would be well suited can be selected from the group consisting of adrenal cortical carcinomas, angiomatoid fibrous histiocytomas (AFH), squamous cell bladder carcinomas, urothelial carcinomas, bone tumours, e.g. adamantinomas, aneurysmal bone cysts, chondroblastomas, chondromas, chondromyxoid fibromas, chondrosarcomas, fibrous dysplasias of the bone, giant cell tumours, osteochondromas or osteosarcomas, breast tumours, e.g. secretory ductal carcinomas, chordomas, clear cell hidradenomas of the skin (CCH), colorectal adenocarcinomas, carcinomas of the gallbladder and extrahepatic bile ducts, combined hepatocellular and cholangiocarcinomas, fibrogenesis imperfecta ossium, pleomorphic salivary gland adenomas head and neck squamous cell carcinomas, chromophobe renal cell carcinomas, clear cell renal cell carcinomas, nephroblastomas (Wilms tumor), papillary renal cell carcinomas, primary renal ASPSCR1-TFE3 t(X;17)(p11;q25) tumors, renal cell carcinomas, laryngeal squamous cell carcinomas, liver adenomas, hepatoblastomas, hepatocellular carcinomas, non-small cell lung carcinomas, small cell lung cancers, malignant melanoma of soft parts, medulloblastomas, meningiomas, neuroblastomas, astrocytic tumours, ependymomas, peripheral nerve sheath tumours, neuroendocrine tumours, e.g. phaeochromocytomas, neurofibromas, oral squamous cell carcinomas, ovarian tumours, e.g. epithelial ovarian tumours, germ cell tumours or sex cord-stromal tumours, pericytomas, pituitary adenomas, posterior uveal melanomas, rhabdoid tumours, skin melanomas, cutaneous benign fibrous histiocytomas, intravenous leiomyomatosis, aggressive angiomyxomas, liposarcomas, myxoid liposarcomas, low grade fibromyxoid sarcomas, soft tissue leiomyosarcomas, biphasic synovial sarcomas, soft tissue chondromas, alveolar soft part sarcomas, clear cell sarcomas, desmoplastic small round cell tumours, elastofibromas, Ewing's tumours, extraskeletal myxoid chondrosarcomas, inflammatory myofibroblastic tumours, lipoblastomas, lipoma, benign lipomatous tumours, liposarcomas, malignant lipomatous tumours, malignant myoepitheliomas, rhabdomyosarcomas, synovial sarcomas, squamous cell cancers, subungual exostosis, germ cell tumours in the testis, spermatocytic seminomas, anaplastic (undifferentiated) carcinomas, oncocytic tumours, papillary carcinomas, carcinomas of the cervix, endometrial carcinomas, leiomyoma as well as vulva and/or vagina tumours. In an embodiment of the invention, the cancer is a lung adenocarcinoma, a prostate adenocarcinoma, a breast ductal cancer or a breast carcinoma, NOS.

As used herein, the term “metastasis” refers to the spread of cancer cells from one organ or body part to another area of the body, i.e. to the formation of metastases. This movement of tumor growth, i.e. metastasis or the formation of metastases, occurs as cancer cells break off the original tumor and spread e.g. by way of the blood or lymph system. Without wishing to be bound by theory, metastasis is an active process and involves an active breaking from the original tumor, for instance by protease digestion of membranes and or cellular matrices, transport to another site of the body, for instance in the blood circulation or in the lymphatic system, and active implantation at said other area of the body. In one embodiment, the cancer is a MST3-dependent cancer. MST3-dependent cancers are cancers where MST3 has become an essential gene. MST3-dependent cancers can be easily identified by depleting the cells of MST3 expression, and identifying the cancers that are not able to grow, migrate or forming metastases in the absence of it.

The present invention also provides a method of screening compounds to identify those which might be useful for treating cancer in a subject by inhibiting MST3 as well as the so-identified compounds. Serine/threonine-protein kinase 24 (STE20 homolog, yeast), also known as MST3, serine/threonine kinase 24, sterile 20-like kinase 3, STK3, STE20-like kinase 3, MST3B, STE20-like kinase MST3, STE20, Mammalian STE20-like protein kinase 3, MST-3, or EC 2.7.11.1, is an enzyme that in humans is encoded by the STK24 gene (isoform b: MAHSPVQSGLPGMQNLKADPEELFTKLEKIGKGSFGEV-FKGIDNRTQKVVAIKIIDLEEAEDEIEDIQQEITVLSQCDSPYVTKYYGSYLKDTKLWIIMEYLGGGSALD LLEPGPLDETQIATILREILKGLDYLHSEKKIHRDIKAANVLLSEHGEVKLADFGVAGQLTDTQIKRNTFV GTPFWMAPEVIKQSAYDSKADIWSLGITAIELARGEPPHSELHPMKVLFLIPKNNPPTLEGNYSKPLKE FVEACLNKEPSFRPTAKELLKHKFILRNAKKTSYLTELIDRYKRWKAEQSHDDSSSEDSDAETDGQAS GGSDSGDWIFTIREKDPKNLENGALQPSDLDRNKMKDIPKRPFSQCLSTIISPLFAELKEKSQACGGNL GSI EELRGAIYLAEEACPGISDTMVAQLVQRLQRYSLSGGGTSSH or isoform a: MDSRAQLWGLALN-KRRATLPHPGGSTNLKADPEELFTKLEKIGKGSFGEVFKGIDNRTQKVVAIKIIDLEEAEDEIEDIQQEIT VLSQCDSPYVTKYYGSYLKDTKLWIIMEYLGGGSALDLLEPGPLDETQIATILREILKGLDYLHSEKKIHR DIKAANVLLSEHGEVKLADFGVAGQLTDTQIKRNTFVGTPFWMAPEVIKQSAYDSKADIWSLGITAIELA RGEPPHSELHPMKVLFLIPKNNPPTLEGNYSKPLKEFVEACLNKEPSFRPTAKELLKHKFILRNAKKTS YLTELIDRYKRWKAEQSHDDSSSEDSDAETDGQASGGSDSGDWIFTIREKDPKNLENGALQPSDLDR NKMKDIPKRPFSQCLSTIISPLFAELKEKSQACGGNLGSIEELRGAIYLAEEACPGISDTMVAQLVQRLQ RYSLSGGGTSSH; SEQ ID NO:1 and 2). The yeast ‘Sterile 20’ gene (STE20) functions upstream of the mitogen-activated protein kinase (MAPK) cascade. In mammals, protein kinases related to STE20 can be divided into 2 subfamilies based on their structure and regulation. Members of the PAK subfamily contain a C-terminal catalytic domain and an N-terminal regulatory domain that has a CDC42 (MIM 116952)-binding domain. In contrast, members of the GCK subfamily, also called the Sps1 subfamily, have an N-terminal catalytic domain and a C-terminal regulatory domain without a CDC42-binding domain. STK24 belongs to the GCK subfamily of STE20-like kinases.

Serine/threonine-protein kinase 38, also known as NDR1, nuclear Dbf2-related, NDR1, NDR1 protein kinase, NDR1, Nuclear Dbf2-related kinase 1, Ndr Ser/Thr kinase-like protein, or EC 2.7.11., is an enzyme that in humans is encoded by the STK38 gene.

Serine/threonine-protein kinase 38 like, also known as NDR2, nuclear Dbf2-related 2, NDR2 protein kinase, NDR2, Nuclear Dbf2-related kinase 2 or EC 2.7.11. 1., is an enzyme that in humans is encoded by the STK38L gene.

The Hippo signaling pathway controls tissue growth in animals. Those animals that lack the pathway have overgrown body parts; the name Hippo comes from the ‘hippopotamus-like’ phenotype produced in Drosophila melanogaster. Known components of the pathway in D. melanogaster include: The receptor Fat, a cadherin which activates the pathway, and also seems to regulate planar cell polarity; Merlin (Mer), an adaptor protein; and Expanded (Ex), an adaptor protein which acts in parallel with Mer; Hippo (Hpo) and Warts (Wts) kinases; and Yorkie (Yki), a transcription factor which upregulates genes responsible for cell proliferation and survival. The pathway has been shown to be conserved in mammalian systems, where it regulates the activity of the YAP proto-oncogene.

The c-myc proto-oncogene, also known as v-myc avian myelocytomatosis viral oncogene homolog, avian myelocytomatosis viral oncogene homolog, bHLHe39 or Transcription factor p64, encodes for a transcription factor of the basic helix-loop-helix family. The protein encoded by this gene is a multifunctional, nuclear phosphoprotein that plays a role in cell cycle progression, apoptosis and cellular transformation. It functions as a transcription factor that regulates transcription of specific target genes. Mutations, overexpression, rearrangement and translocation of this gene have been associated with a variety of hematopoietic tumors, leukemias and lymphomas, including Burkitt lymphoma. There is evidence to show that alternative translation initiations from an upstream, in-frame non-AUG (CUG) and a downstream AUG start site result in the production of two isoforms with distinct N-termini. c-myc abundance is regulated on multiple levels, including expression of mRNA, translation and protein stability.

Cyclin-dependent kinase inhibitor 1A (p21, Cip1), also known as CDKN1A, is a protein which in humans is encoded by the CDKN1A gene located on chromosome 6 (6p21.2). This gene encodes a potent cyclin-dependent kinase inhibitor (CKI). The encoded protein binds to and inhibits the activity of cyclin-CDK2 or -CDK4 complexes, and thus functions as a regulator of cell cycle progression at G1. The expression of this gene is tightly controlled by the tumor suppressor protein p53, through which this protein mediates the p53-dependent cell cycle G1 phase arrest in response to a variety of stress stimuli. This protein can interact with proliferating cell nuclear antigen (PCNA), a DNA polymerase accessory factor, and plays a regulatory role in S phase DNA replication and DNA damage repair. This protein was reported to be specifically cleaved by CASP3-like caspases, which thus leads to a dramatic activation of CDK2, and may be instrumental in the execution of apoptosis following caspase activation. Two alternatively spliced variants, which encode an identical protein, have been reported. p21 is a CKI that directly inhibits the activity of cyclin-CDK2 and cyclin-CDK4 complexes. p21 functions as a regulator of cell cycle progression at S phase. The expression of p21 is controlled by the tumor suppressor protein p53. Sometimes, it is expressed without being induced by P53. This kind of induction plays a big role in p53 independent apoptosis by p21. Expression of p21 is mainly dependent on two factors 1) stimulus provided 2) type of the cell. The function of this gene relates in part to stress response. p21 is the major transcriptional target of the tumor suppressor gene, p53; despite this, loss-of-function mutations in p21 (unlike p53) do not accumulate in cancer nor do they predispose to cancer incidence. In fact, mice genetically engineered to lack p21 develop rather normally and are not susceptible to cancer at a higher rate than the norm (again, unlike p53). p21 also mediates the resistance of hematopoietic cells to an infection with HIV by complexing with the HIV integrase and thereby aborting chromosomal integration of the provirus.

Cyclin-dependent kinase inhibitor 1B (p27, Kip1), also known as CDKN1B, is a human gene. It encodes a protein which belongs to the Cip/Kip family of cyclin dependent kinase (Cdk) inhibitor proteins. The encoded protein binds to and prevents the activation of cyclin E-CDK2 or cyclin D-CDK4 complexes, and thus controls the cell cycle progression at G1. It is often referred to as a cell cycle inhibitor protein because its major function is to stop or slow down the cell division cycle. The p27Kip1 gene has a DNA sequence similar to other members of the “Cip/Kip” family which include the p21Cip1/Waf1 and p57Kip2 genes. In addition to this structural similarity the “Cip/Kip” proteins share the functional characteristic of being able to bind several different classes of Cyclin and Cdk molecules. For example, p27Kip1 binds to cyclin D either alone, or when complexed to its catalytic subunit CDK4. In doing so p27Kip1 inhibits the catalytic activity of Cdk4, which means that it prevents Cdk4 from adding phosphate residues to its principal substrate, the retinoblastoma (pRb) protein. Increased levels of the p27Kip1 protein typically cause cells to arrest in the G1 phase of the cell cycle. Likewise, p27Kip1 is able to bind other Cdk proteins when complexed to cyclin subunits such as Cyclin E/Cdk2 and Cyclin A/Cdk2. In general, extracellular growth factors which prevent cell growth cause an increase in p27Kip1 levels inside a cell. For example, levels of p27Kip1 increase when Transforming Growth Factor r3 (TGF) is present outside of epithelial cells causing a growth arrest. In contrast interleukin 2 (IL-2) causes p27Kip1 levels to drop in T-lymphocytes. A mutation of this gene may lead to loss of control over the cell cycle leading to uncontrolled cellular proliferation.

Cyclin-dependent kinase inhibitor 10 (p57, Kip2), also known as CDKN1C, is protein which in humans is encoded by the CDKN1C imprinted gene. Cyclin-dependent kinase inhibitor 10 is a tight-binding inhibitor of several G1 cyclin/Cdk complexes and a negative regulator of cell proliferation. Mutations of CDKN1C are implicated in sporadic cancers and Beckwith-Wiedemann syndrome suggesting that it is a tumor suppressor candidate. CDKN1C is a tumor suppressor human gene on chromosome 11 (11p15) and belongs to the cip/kip gene family. It encodes a cell cycle inhibitor that binds to G1 cyclin-CDK complexes. Thus p57KIP2 causes arrest of the cell cycle in G1 phase. A mutation of this gene may lead to loss of control over the cell cycle leading to uncontrolled cellular proliferation. p57KIP2 has been associated with Beckwith-Wiedemann syndrome (BWS) which is characterized by increased risk of tumor formation in childhood.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

### Materials and Methods

Cell Culture, Transfections and Treatments:

HeLa, HeLa S3, HEK293, U20S and HCT116 cells were maintained in DMEM supplemented with 10% fetal calf serum (FCS). Cells were transfected using Fugene 6 (Roche), Lipofectamine-2000 (Invitrogen) or jetPEI (Polyplus Transfection) as described by the manufacturer. For siRNA mediated knock-down of MST3 or p21 cells were transfected with pre-designed siRNA (Qiagen) using Lipofectamine-2000. Validated control siRNAs were from Qiagen and used according to the manufacturers instructions. For rescue experiments targeting elevated p21 level, cells were transfected twice in 24 h intervals with siRNA against p21 or control siRNA. HeLa cells expressing tetracycline inducible shRNA against NDR1 and NDR2 and U20S cells stably expressing shRNA against NDR1 together with an NDR1 wt rescue construct have been described elsewhere (A. Hergovich et al., Mol Cell 25, 625-634 (2007).; A. Vichalkovski et al., Curr Biol 18, 1889-1895 (2008)). HeLa and U20S cells stably expressing shRNA against NDR1 or NDR2 alone were generated as described in (A. Hergovich et al., Mol Cell 25, 625-634 (2007).; A. Vichalkovski et al., Curr Biol 18, 1889-1895 (2008)). To access protein stability, cells were treated with 50 μg/ml cycloheximide (CHX) or 10 μM MG132 for the indicated time. Staining for cellular senescence was performed as described in (G. P. Dimri et al., Proc Natl Acad Sci USA 92, 9363-9367 (1995)).

Reagents and Antibodies:

The generation of antibodies against T-444-P, NDR1, NDR2 and NDR1/2 have previously been described (A. Vichalkovski et al., Curr Biol 18, 1889-1895 (2008)). Antibodies against Cyclin A, Cyclin E, Cyclin B1, cdc2, p27, gfp, c-myc (N262), HA (Y11) and actin were from Santa Cruz. Antibodies to detect p21, Cyclin D1, Cdk4 and myc-tagged proteins (71D10) were from Cell Signalling. Antibodies against HA-tag (12CA5, 42F13), Tubulin (Y1/2) and c-myc (9E10) were used as hybridoma supernatants. Additional antibodies used included: anti-P-p21-5146 (Abgent), anti-P-MST4-T178/-MST3-T190/-STK25-T174 (referred to as P-MST3; Epitomics), anti-MST3 (BD Bioscience) and anti-FLAG (M2) (Sigma). Nocodazole, Thymidine, PI and Cycloheximide were from Sigma. SB203580 and SB202190 were from Alexis. MG132 was from Calbiotech and the BrdU and the anti-BrdU antibody was from BD Bioscience.

Construction of Plasmids:

The construction of plasmids encoding cDNAs for tagged variants of NDR1, NDR2, MST1, MST2 and MST3 has been described elsewhere (A. Hergovich et al., Mol Cell 25, 625-634 (2007); A. Vichalkovski et al., Curr Biol 18, 1889-1895 (2008); A. Hergovich et al., Mol Cell Biol 25, 8259-8272 (2005)). RNAi-rescue constructs for NDR2 were obtained by introducing silent mutations into the shRNA target sites using PCR-mutagenesis. For constructs expressing cDNAs fused to an IRES-gfp, the IRES-gfp cassette was excised from the pMIG-vector (a kind gift from W. Hahn, Dana-Farber Cancer Institute, Boston) using XhoI/SalI digestion and inserted into pcDNA3 containing the indicated cDNAs using XhoI. Constructs for pGEX2T-GSTp21, pcDNA3-p21 and pcDNA-myc-p21 were obtained by PCR cloning attaching BamHI/XhoI sites to p21-cDNA (a kind gift from N. Lamb, Institut de Genetique Humaine, Montpellier) and insertion into the BamHI/XhoI sites of the respective vector. Mutation of T145, S146 and T145/S146 to alanine was done by PCR-mutagenesis. cDNA encoding c-myc was a kind gift from N. Hynes (Friedrich-Miescher Institute for Biomedical Research, Basel) and HA-tagged c-myc was obtained similar to myc-p21 by PCR-cloning into a pcDNA3-HA vector. HA-tagged variants of c-myc containing only the first 215 amino-acids (c-myc-AC) or the last 234 amino-acids (c-myc-ΔN) were obtained by PCR cloning. Deletion of the MB1 or MB2 domain was performed by mutagenesis-PCR. Primer sequences are available upon request. Vectors encoding cDNA for Skp2 or ubiquitin (Ub) were kind gifts from W. Krek (Institute of Cell Biology, ETH Zürich, Zürich) and W. Filipowicz (Friedrich-Miescher Institute for Biomedical Research, Basel).

Protein Extraction, Immunoprecipitation, Immunoblotting and Ubiquitination Analysis:

Proteins extraction from cultured cells, immunoprecipitation and immunoblotting were done as described previously (A. Hergovich et al., Mol Cell Bio125, 8259-8272 (2005)). The following antibodies were used for immunoprecipitation: anti-HA (12CA5), anti-c-myc (9E10, N262) and a mixture of NDR1 and NDR2 specific antibodies to access endogenous NDR species. For quantification using the Licor Odyssey System, western blots were incubated with secondary antibodies conjugated with fluorescent dyes. Quantifications were carried out using the Licor Odyssey software. Analysis of c-myc ubiquitination was performed as described in (Cold Spring Herb. protocols; 2006, doi:10.1101/pdb.prot4616).

Cell Cycle Analysis:

HeLa and HeLa S3 cells were synchronized using either a double thymidine block with subsequent nocodazole arrest and mitotic shake-off (L. A. Tintignac et al., Mol Cell Bio124, 1809-1821 (2004)) or a single treatment with 100 ng/mlnocodazole for 14 h. Cells were washed free from nocodazole with ice-cold PBS and released into fresh medium for the indicated time before harvesting. Cell cycle distribution was accessed using either BrdU labeling, as described by the manufacturer or PI staining as described (A. Hergovich et al., Mol Cell 25, 625-634 (2007)). To detect cells blocked in G1a method described in (K. Mikule et al., Nat Cell Biol 9, 160-170 (2007)) was used. In short, cells were seeded at defined densities into 10 cm dishes. 24 h later 2.5 μg/ml nocodazole was added for 14-16 h to terminally arrest cells at G2/M border. Cells were harvested by trypsination and processed for FACS analysis.

Proliferation Assays:

For the analysis of cell proliferation, cells were seeded at defined densities in triplicates, for experiments including inducible shRNAs fresh tetracycline was added each day starting with cell seeding. After the indicated time, cells were harvested by trypsination and counted using a ViCell-automated cell counter (Beckman-Coulter).

RNA Isolation and Quantitative Real Time PCR:

Total RNA from cells was isolated with TRIzol reagent (Invitrogen) and further purified using RNeasy kit (Qiagen). cDNA from samples was generated from 2 μg of total RNA using M-MuLV reverse transcriptase (NEB) and Oligo-dT primers. Quantitative RT-PCR to detect p21, p27 and c-myc (primer sequences upon request) was carried out using SYBR green technology in an ABI Prism 7000 detection system (Applied Biosystems).

Mammalian NDR kinases are implicated in the regulation of cell cycle-dependent processes such as centrosome duplication and the alignment of mitotic chromosomes (Chiba, S. et al., Curr Biol 19, 675-681 (2009); Hergovich, A. et al., Curr Biol 19, 1692-1702 (2009)). To better define cell cycle function(s) of NDR kinases, the present inventors analyzed whether NDR kinase activity changes during cell cycle progression. Hydrophobic motif (HM) phosphorylation of NDR1 and NDR2, as an indicator of NDR kinase activity, was nearly absent in M phase, increased 3 h after mitotic shake-off upon entry into G1 phase and peaked around 6-8 h in G1 phase. Activation of NDR persisted into S phase (12-14 h) and started to decrease 14 h after shake-off. Analysis of cell cycle markers and FACS staining confirmed that NDR activation peaked in G1 phase with activation persisting into S phase. G1-activation of NDR1/2 was confirmed by analyzing endogenous NDR1/2 activity using a peptide kinase assay. Since three members of the mammalian Step 20-like kinases (MST1/2/3) can regulate NDR kinases (Chiba, S. et al., Curr Biol 19, 675-681 (2009); Hergovich, A. et al., Curr Biol 19, 1692-1702 (2009); Stegert, M. R. et al., Molecular and cellular biology 25, 11019-11029 (2005); Vichalkovski, A. et al., Curr Biol 18, 1889-1895 (2008)), the present inventors tested whether MST kinases are important for NDR1/2 activation in G1. To this end, they analyzed NDR phosphorylation in G1 upon overexpression of dominant negative (DN) variants of MST1-3. Although overexpression of DN-MST1 and DN-MST2 hardly affected NDR activation, DN-MST3 expression significantly reduced NDR phosphorylation in this setting. This finding was confirmed using siRNA-mediated depletion of MST3. Interestingly, they also observed an increase in phosphorylated MST3 in G1 phase cells versus M phase-arrested cells, indicating that MST3 activity is increased in G1 phase of the cell cycle. Collectively, these results revealed that NDR kinases were activated in G1 phase of the cell cycle, with the activation persisting into S phase. Furthermore, our experiments revealed MST3 as the responsible upstream kinase for NDR1/2 in this setting, providing the first functional link between NDR1/2 and MST3.

To analyze whether NDR kinases functioned in cell cycle progression and proliferation the present inventors generated HeLa cells expressing inducible shRNA against NDR1 and NDR2. Knock-down of NDR kinases consistently resulted in decreased proliferation of around 50%, which were not observed in control clones expressing shRNA against firefly luciferase. Reduced proliferation in NDR-depleted cells was accompanied by an increase in cells in G1 and a decrease in cells in S phase. G1 phase arrest was confirmed by treating cells with nocodazole to accumulate cycling cells at the G2/M border. The resent inventors analyzed the mechanisms underlying the G1-block by investigating expression levels of known G1/S regulators. Interestingly, the expression of p21 and p27 was elevated in NDR1/2-knock-down cells without significant decrease in the expression of Cyclins and Cdks. In addition, the expression of the c-myc proto-oncogene was reduced. This suggested that the observed G1-block upon depletion of NDR1/2 was due to the inhibition of Cyclin-Cdk complexes by increased levels of p21 and p27.

It has been shown that c-myc is able to repress p21 and p27 expression (Claassen, G. F. & Hann, S. R., Proceedings of the National Academy of Sciences of the United States of America 97, 9498-9503 (2000); Yang, W. et al.; Oncogene 20, 1688-1702 (2001)); therefore the present inventors tested whether depletion of NDR1/2 would result in increased expression of p21 and p27 mRNAs. Strikingly, although p27 mRNA levels were clearly increased, they did not observe any elevation of p21 mRNA. In addition, depletion of NDR did not affect c-myc mRNA expression suggesting that NDR kinases regulated the protein levels of c-myc.

A report analyzing post-transcriptional modifiers of c-myc in human B-cells implicated NDR1 in the regulation of c-myc protein stability (Wang, K. et al.; Nature biotechnology 27, 829-839 (2009)). In full agreement with this report, c-myc protein levels were rescued by the addition of the proteasomal inhibitor MG132 in NDR1/2 depleted HeLa cells. In addition, the experiments of the inventors confirmed an interaction between NDR1 and c-myc both on overexpressed and endogenous levels. Furthermore, NDR2 bound to c-myc with similar affinity as NDR1. Next, the present inventors analyzed the determinants for NDR binding to c-myc. Using co-immunoprecipitation experiments, they found that c-myc interacted mainly with the N-terminal region (NTR; residues 1-82) of NDR1. In addition, interaction was shown to be modulated by HM-phosphorylation (Thr444). Both the NTR and the HM-phosphorylation site have been shown to be essential for NDR full kinase activity (Stegert, M. R. et al., Molecular and cellular biology 25, 11019-11029 (2005); Millward, T. A. et al.; The EMBO journal 17, 5913-5922 (1998)), suggesting that NDR might bind to c-myc in an active conformation. Nevertheless, NDR1 kinase dead (kd) associated with c-myc similarly to NDR1 wt. In addition, both NDR1 wt and NDR1 kd significantly stabilized c-myc levels. NDR1 mutants defective in c-myc interaction had minor effects on c-myc stability. Furthermore, increasing HM-phosphorylation of NDR by coexpression of MST3 increased complex formation and c-myc stability. In addition to stabilizing c-myc protein levels, NDR1 wt but not NDR1TA (T444A) overexpression stimulated c-myc mediated transcription. Finally, the effects of overexpression and HM-phosphorylation of NDR1 on endogenous c-myc levels were tested. Strikingly, overexpression of NDR1 wt and NDR1 kd increased endogenous c-myc levels, which could be further increased by stimulating HM-phosphorylation by coexpression of MST3.

Degradation of c-myc is tightly regulated by the ubiquitin-proteasome system (Vervoorts, J. et al.; The Journal of biological chemistry 281, 34725-34729 (2006)); hence the present inventors tested whether NDR expression affected c-myc ubiquitination. Indeed, they observed that the stabilizing effect of NDR1 overexpression on c-myc was due to impaired c-myc ubiquitination. Two E3 ubiquitin ligases, FBW7 and Skp2, have been shown to regulate c-myc ubiquitination and degradation (Vervoorts, J. et al.; The Journal of biological chemistry 281, 34725-34729 (2006)). Whereas interaction of FBW7 with c-myc is regulated by phosphorlyation of the MBI domain, Skp2 binds to the MBII domain of c-myc. The present inventors tested the effect of NDR overexpression on Skp2 and FBW7 mediated ubiquitination. Surprisingly, ubiquitination of c-myc by both FBW7 and Skp2 was inhibited by NDR1 wt overexpession, although NDR1 did not compete with either FBW7 or Skp2 for c-myc interaction, although, they found that NDR1 bound to the MBII domain of c-myc. This indicates a different mechanism by which NDR inhibits c-myc ubiquitination. Collectively, the inventor's analysis suggests a role for NDR kinases in the regulation of c-myc protein stability by interfering with Skp2 and FBW7 mediated ubiquitination. Interestingly, NDR kinases interacted with c-myc dependent on HM-phosphorylation, but independent of NDR kinase activity.

To confirm the effects of NDR on c-myc, the present inventors performed experiments to rescue the effects of depletion of NDR2 by transient overexpression of NDR2 mutants refractory to shRNA. Indeed, overexpression of NDR2 wt and NDR2 kd in this setting rescued the effects on c-myc. Next, they analyzed whether altered p21 or c-myc levels mediated the G1-arrest observed in NDR-depleted cells. Interestingly, whereas forced overexpression of c-myc upon NDR1 knock down did not overcome the G1-arrest, it repressed the increase in p27 levels. Restoring c-myc levels failed to rescue cells from shNDR1/2 induced G1-arrest, but their results suggested that NDR kinases could promote G1-progression/S phase entry by stabilizing c-myc. Strikingly, NDR overexpression in cells released from quiescence (G0) not only resulted in increased c-myc levels, but also promoted S phase entry. Importantly, NDR activation in this setting was increased in G1 and declined in S phase, fully confirming the results obtained in M phase-arrested cells. To confirm the effect of NDR overexpression on c-myc mediated cell cycle progression we made use of a Rat1 cell line expressing a c-myc-ER construct (Littlewood, T. D et al.; Nucleic acids research 23, 1686-1690 (1995)). Importantly, overexpression of NDR not only resulted in slightly increased steady state levels of the c-myc-ER construct, but also promoted c-myc dependent S phase entry.

Taken together, the two signaling mechanisms downstream of NDR kinases defined in this disclosure can be summarized as follows: Whereas knock-down of NDR1/2 resulted in G1-arrest dependent on increased p21 stability, overexpression of NDR kinases increased S phase entry by stabilizing c-myc. These findings implicate a novel MST3-NDR-c-myc/p21 axis as new signaling module regulating G1/S-transition. Previous reports revealed a role for NDR kinases in regulating mitotic chromosome alignment, centrosome duplication and apoptosis. However, in these contexts NDR kinases have been shown to function downstream of MST1 and MST2, which have been established as tumor suppressors as part of the HIPPO pathway (Zhao, B. et al.; Current opinion in cell biology 20, 638-646 (2008)). The present results thus indicate a potential dual role for NDR kinases in regulating cell proliferation and apoptosis. Whereas NDR kinases function as tumor suppressors by promoting apoptosis downstream of MST1/2, the newly established MST3-NDR axis promotes cell proliferation by restricting p21 levels and stabilizing the proto-oncogene c-myc. Therefore, the present work provides a platform for establishing a dual and most likely cell context-dependent role for NDR kinases in normal and cancer cell biology.

1. A method for modulating miRNA in a sample, said method being characterized in that the sample is contacted with a modulator of XRN1.

2. (canceled)

3. The method of claim 16, wherein the method is performed to treat a disease and wherein a therapeutically effective amount of said modulator of XRN1 is administered to said subject.

4. The method of claim 3, wherein the disease is a cancer, a metabolic disease, a developmental disorder, a cardiac disease or a viral infection.

5. The method of claim 16, wherein the modulator of XRN1 is a small molecule, for instancc a RNasc inhibitor.

6. The method of claim 16, wherein the modulator of XRN1 is an antibody.

7. The method of claim 16, wherein the modulator of XRN1 is an agonist.

8. The method of claim 16, wherein the modulator of XRN1 is an inhibitor of XRN1.

9. The method of claim 8 wherein said inhibitor of XRN1 decreases or silences the expression of XRN1.

10. The method of claim 9 wherein the inhibitor is a siRNA.

11. The method of claim 16, wherein the subject is a mammal.

12. (canceled)

13. (canceled)

14. A method for the identification of a substance that modulates the expression of XRN1 and/or its biological activity, which method comprises the steps of:
(i) contacting a XRN1 polypeptide or a fragment thereof having the biological activity of XRN1, a polynucleotide encoding such a polypeptide or polypeptide fragment, an expression vector comprising such a polynucleotide or a cell comprising such an expression vector, and a test substance under conditions that in the absence of the test substance would permit XRN1 expression and/or biological activity; and
(ii) determining the amount of expression and/or biological activity of XRN1, e.g. the degradation of mature miRNA, to determine whether the test substance modulates biological activity and/or expression of XRN1, wherein a test substance which modulates biological activity and/or expression of the XRN1 is a potential therapeutical agent to treat cancer.

15. (canceled)

16. A method of modulating miRNA in a subject, the method comprising administering an effective amount of a modulator of XRN1 to said subject.

17. The method of claim 5, wherein the small molecule is RNase inhibitor.

18. The method of claim 11, wherein the mammal is a human.

19. A method of modulating the efficiency of RNAi activity in a sample, the method comprising contacting the sample with a modulator of XRN1 and/or XRN2.
